(19) United States
(12) Patent Application Publication
(10) Pub. No.: US 2012/0277614 A1
(43) Pub. Date: Nov. 1, 2012

(54) High-Frequency Oscillatory Ventilation Monitoring Method and System

(76) Inventors: Benjamin Horowitz, Pelham, NY (US); Robert M. Handzel, Liverpool, NY (US); Megan M. Mekarski, East Amherst, NY (US); William H. Sipprell, Hamburg, NY (US); Patricia R. Chess, Rochester, NY (US); Timothy P. Stevens, Pittsford, NY (US); Scott Seidman, Fairport, NY (US)

(21) Appl. No.: 13/391,285

(22) PCT Filed: Aug. 19, 2010
(86) PCT No.: PCT/US10/46034

§ 371 Date: Jun. 29, 2012

Related U.S. Application Data
(60) Provisional application No. 61/235,348, filed on Aug. 19, 2009.

(51) Int. Cl. A61B 5/11

(52) U.S. Cl. 600/534

(57) ABSTRACT

A method of monitoring high frequency oscillatory ventilation (HFOV) wherein the oscillatory movement of the chest wall of an individual is measured. An average amplitude is determined and compared to a pre-determined baseline amplitude, which is established by using the average amplitude at a particular instant of time. If the variance between the average amplitude and the baseline meets and/or exceeds a pre-determined threshold above or below the baseline value, an operator is alerted.

### CROSS-REFERENCE TO RELATED APPLICATION

This application claims the benefit of priority to U.S. provisional patent application Ser. No. 61/235,348, filed on Aug. 19, 2009, now pending, the disclosure of which is incorporated herein by reference.

### FIELD OF THE INVENTION

The present invention relates generally to monitoring artificial ventilation, and in particular, to monitoring the high frequency oscillatory ventilation (HFOV) of an individual.

### BACKGROUND OF THE INVENTION

Premature babies often require ventilation because of an underdeveloped respiratory system. Traditional ventilation provides high volumes of air to the lungs at a rate similar to natural breathing (12 breaths per minute). This is often not the most appropriate option for neonates because the high volumes of forced air can overextend the infant's fragile lung tissues. Instead, high frequency oscillatory ventilation (HFOV) is used in the case of infants with underdeveloped lungs. HFOV operates on an open lung strategy and does not fully extend or collapse the alveoli of the lungs. It provides much smaller volumes of air at a much faster rate (600 breaths per minute) to still offer proper ventilation. Both types of ventilators use intubation to provide the patients with respiratory gas exchange.

A problem with the HFOV in neonates is that it is easy for the tubing to become blocked or move out of place. When this occurs, the patient is not being sufficiently ventilated, which could lead to serious medical complications. There is currently no incorporated alarm system to detect a blockage or improper placement. Because of the extremely low volume of air each oscillation for small individuals (e.g. neonates, small animals), currently used methodologies of measuring tidal volume are not practical. Chest wall vibration is considered an indicator of ventilation on HFOV. Visual inspection of chest wall movement (“excursion”) by medical staff is presently the only immediate method of evaluating proper ventilation. Visual inspection is subjective and imprecise, and evaluation can vary between staff. There are no current, practical solutions to measure chest wall movement in small individuals. Therefore, it is desirable to have an objective, automated monitoring system of neonates on HFOV.

### BRIEF SUMMARY OF THE INVENTION

A method of monitoring high frequency oscillatory ventilation (HFOV) wherein the oscillatory movement of the chest wall of an individual is measured. The frequency of the oscillation is determined by an operator of the HFOV oscillator (the “oscillator”). The chest wall excursion is measured by an accelerometer. The amplitude of each chest wall excursion is determined and a plurality of amplitudes is averaged to determine an average amplitude over a pre-determined period of time (an “averaging window”). The averaging window may be a moving window, where a moving average amplitude may be continuously calculated from the most recent data.

The average amplitude is then compared to a pre-determined baseline amplitude, which may be established by using the average amplitude at a particular instant of time. As such, when an individual is placed on an oscillator, the operator can establish a baseline average amplitude. The individual will then be monitored for variance against this baseline. If the variance meets and/or exceeds a pre-determined threshold above or below the baseline value, the operator is alerted.

A system according to another embodiment of the invention comprises an accelerometer. The accelerometer comprises a printed circuit board (“PCB”) and a low-pass filter. The accelerometer is fixed relative to a position of the body of the individual. The accelerometer measures the oscillatory movement of the chest wall of the individual caused by HFOV. The accelerometer transmits a signal to a monitor configured to receive the signal. The monitor has a signal processor configured to derive an average amplitude of the oscillations of the signal.

The system calculates a time-based average of the amplitude and compares the average amplitude to a baseline value to determine a variance. The system alerts an operator (e.g. audible and/or visible alarm(s)) if the variance is greater than a predetermined threshold.

### DESCRIPTION OF THE DRAWINGS

For a fuller understanding of the nature and objects of the invention, reference should be made to the following detailed description taken in conjunction with the accompanying drawings, in which:

FIG. 1 is a method according to an embodiment of the invention

FIG. 2a depicts various embodiments of a printed circuit board for an accelerometer of the present invention;

FIG. 2b is a schematic of an embodiment of an accelerometer according to the present invention;

FIG. 3 depicts various examples of accelerometers having differing sizes and a U.S. quarter for size comparison;

FIG. 4 is a schematic of the circuitry of a portion of a system according to another embodiment of the invention;

FIG. 5 is a signal trace of the signal measured at positions in the circuit of FIG. 4;

FIG. 6 depicts a control panel of a system according to the present invention;

FIG. 7 depicts a side panel of the system of FIG. 6; and

FIG. 8 is a system level diagram of a system according to another embodiment of the invention;

FIG. 9 is a graph showing test results of a system according to the present invention.

### DETAILED DESCRIPTION OF THE INVENTION

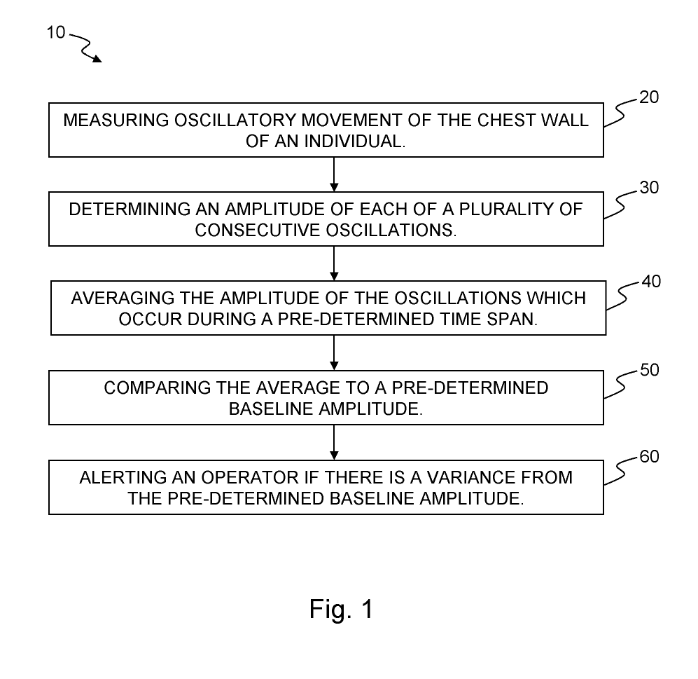

FIG. 1 depicts a method 10 of monitoring high frequency oscillatory ventilation (HFOV) according to an embodiment of the present invention wherein the oscillatory movement of the chest wall of an individual is measured 20. The individual may be an animal. The individual may be a neonate. HFOV forces air into and out of the lungs of the individual, and such activity causes movement of the chest wall as the lungs expand and contract with air volume. This movement is oscillatory—an oscillator causes air to enter and then exit the lungs repeatedly. The frequency of the oscillation is determined by an operator of the oscillator and is fixed insofar as lung compliance does not affect the frequency. The frequency may be set at, for example, twice the rate of the individual's heart beat. The frequency may be as high as 900 oscillations per minute or higher.

The chest wall excursion is measured by an accelerometer, which measures the acceleration of the chest wall to determine the excursion. Other methods of measuring chest wall excursion may be utilized. Such other methods must be capable of accurately measuring small chest wall excursions, for example, the chest wall excursion of a neonate may be less than 2 mm. The chest wall excursion is a measure of the amplitude of the oscillation of the chest wall. In this way, frequency and amplitude are known values of the chest wall oscillation. The amplitude of each chest wall excursion is determined 30 and a plurality of amplitudes is averaged 40 to determine an average amplitude over a pre-determined period of time (an “averaging window”). For example, if the frequency is 300 oscillations per minute, and the averaging window is 10 seconds, an average amplitude can be calculated from 50 amplitude values. The averaging window may be a moving window. As such, using the example above, a moving average amplitude is continuously calculated from the most recent 10 seconds (50 cycles) of data.

The average amplitude is then compared 50 to a pre-determined baseline amplitude. The baseline amplitude may be determined from operator experience. Alternatively, the baseline amplitude may be calculated from known relationships between chest wall excursion and tidal volume. The baseline amplitude can also be established by way of a monitoring system by causing an average amplitude (e.g. the most recent calculated moving average amplitude) to be “saved” and used as the baseline amplitude. For example, in an embodiment, a system has an input (such as a button) which, when activated by an operator, will cause the then current average amplitude to be the baseline by which subsequent average amplitudes will be compared.

As such, when an individual is placed on an oscillator, the operator (e.g. doctor) typically adjusts the parameters of the HFOV according to methods already known. The operator may then depress a baseline button of a system according to the present invention to establish a baseline amplitude. The individual will then be monitored for variance against this baseline.

If the variance exceeds a pre-determined threshold above or below the baseline value, the operator is alerted 60. For example, an audible and/or visible alarm may be triggered. The variance can be determined as a percentage of the baseline value or as an absolute difference between average amplitude and baseline amplitude. For example, an operator may set a threshold at 110% of the baseline, or the operator may set a threshold at 30 oscillations per minute greater than the baseline value. The thresholds for over-variance and under-variance conditions can be the same or different. For example, an operator may wish to be alerted if the average amplitude is greater than 110% of the baseline, or less than 95% of the baseline.

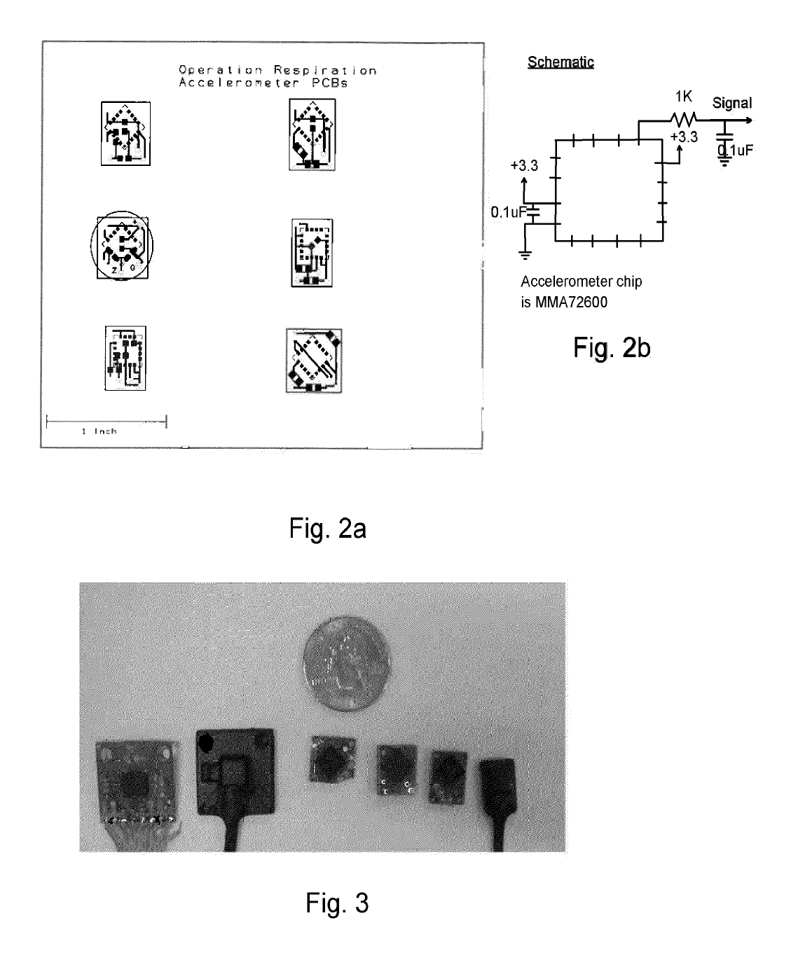

In another embodiment of the present invention, the above-described method is performed by a system for monitoring chest wall excursion, having an accelerometer. The accelerometer comprises an accelerometer chip, which may be a three-axis accelerometer chip, such as, for example, an MMA7260Q. The accelerometer may further comprise a printed circuit board (“PCB”) and a low-pass filter. FIG. 2b shows one example of a circuit which may be utilized in the accelerometer, including an accelerometer chip and components forming a low-pass filter. Such a circuit may be implemented in a PCB having different component arrangements. Six exemplary PCB arrangements are depicted in FIG. 2a. The low-pass circuit is preferably located near the accelerometer chip to reduce the potential for noise in the signal sent from the accelerometer.

The accelerometer is configured to be a size suitable for fixation to the body of an individual. FIG. 3 depicts several accelerometer configurations having different sizes (FIG. 3 also shows a U.S. quarter for relative size comparison). The accelerometer can be of any shape, including, but not limited to, round, square, or rectangular. The PCB may be two-sided, such that the area of the PCB is reduced. For example, the PCB may be configured such that the accelerometer chip is on a first side of the PCB, while the low-pass filter components are a second side of the PCB. The accelerometer may be coated in an insulating coating. The accelerometer may be coated in a coating which is compatible with exposure to skin.

The accelerometer is fixed relative to a position of the body of the individual. As such, the accelerometer measures the oscillatory movement of the chest wall of the individual caused by HFOV. Techniques for fixing a system relative to a body are known in the art and may include adhesives, elastic straps, hook-and-loop fastened straps, and the like. The accelerometer may be fixed against the skin of the individual or on a garment of the individual.

The accelerometer transmits a signal to a monitor configured to receive the signal. The monitor and accelerometer may be connected by a wire over which the signal may be transmitted and received. Alternatively, the accelerometer may further comprise a circuit for wireless communications, such that the monitor and the accelerometer may communicate wirelessly. The monitor further comprises a signal processor configured to derive an average amplitude of the oscillations of the signal.

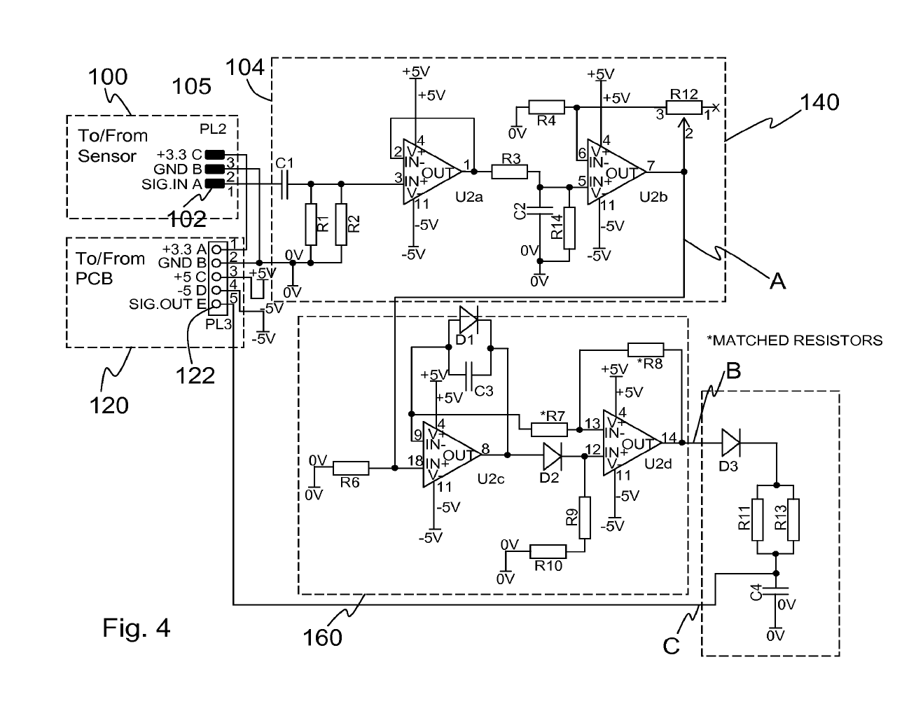

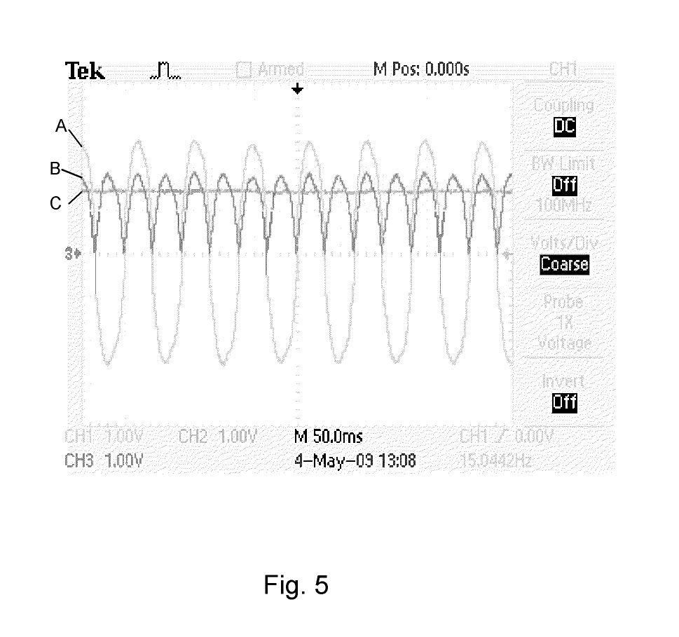

FIG. 4 shows a portion of a signal processor according to one embodiment of the invention. In the figure, boxes are shown to group components for convenience of description. Box 100 shows the input 102 to the circuit from the accelerometer, a power input 104, and a ground connection 106. Box 120 shows an output connector 124 comprising a signal output 122. Box 140 shows circuitry to filter and amplify the signal. Box 160 shows circuitry which rectifies the signal. And box 180 shows circuitry to detect the envelope (roughly, the amplitude) of the signal. It should be noted that envelope detector circuitry (e.g., the circuit of box 180) is optional. In such embodiments, a processing circuit (see below) may operate using the amplitude of the waveform to determine breathing variations. The signal output 122 will have a voltage based on the amplitude of the signal. FIG. 5 shows signal traces recorded on an oscilloscope of the signal at various stages of processing. Trace A depicts the signal from the accelerometer after filtering and amplification (measured at position A in FIG. 4). Trace B depicts the same signal after rectification (measured at position B in FIG. 4). Trace C represents the envelope of the rectified signal (measured at position C in FIG. 4).

A processing circuit may be connected to the output connector 124 to further process the output signal 122. Such a processing circuit may comprise a microcontroller, such as, for example, a PIC18F4685, or a digital signal controller, for example, a dsPIC30F3011. Where the processing circuit comprises a microcontroller, the processing circuit is configured to calculate a time-based average of the amplitude. For example, the processing circuit may calculate an average of the amplitude over a 10-second window. The time window, and thus the average value, may be a moving calculation such that the most recent 10-second average is provided. The processing circuit may be further configured to compare the average amplitude to a baseline value to determine a variance. The processing circuit triggers an alert an operator (e.g. audible and/or visible alarm(s)) if the variance is greater than a predetermined threshold. The processing circuit may also make use of three-axis accelerometer signals to calculate acceleration in three dimensional space. The processing circuit may perform digital signal processing techniques, such as Fast Fourier Transform, digital filtering, or the Goertzel algorithm to isolate and extract the portion of the acceleration signal that best represents chest wall motion in response to HFOV.

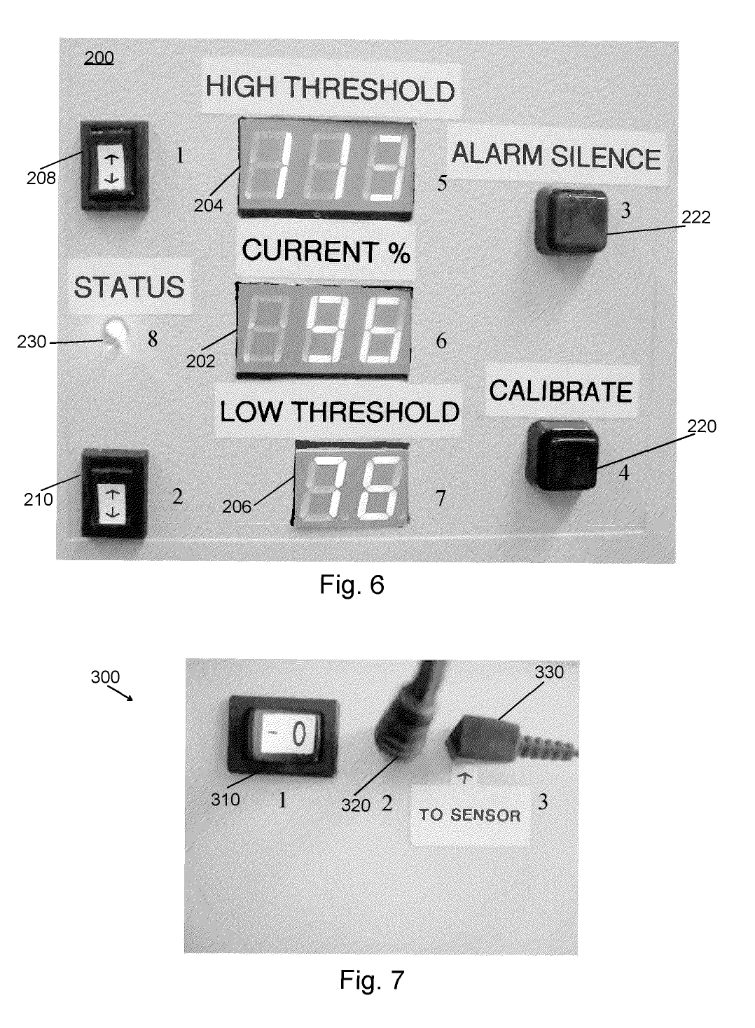

The processing circuit may also control a user interface panel 200, an example of which is shown in FIG. 6. User interface panel 200 comprises a current display 202 which displays the current average amplitude. The current average amplitude may be displayed as a percentage of the baseline value. User interface panel 200 further comprises a high-threshold display 204 and a low-threshold display 206 which display the respective thresholds over- and under- which the operator will be alerted. User interface panel 200 also includes set switches 208, 210 with which an operator sets the high- and low-threshold values. The thresholds may be displayed and set in terms of percentage of the baseline value.

A calibrate button 220 may be provided on the user interface panel 200 by which the operator may cause the processing circuit to set the baseline equal to the current average amplitude. The calibrate button 220 may be a momentary button. An alarm silence button 222 may be provided by which the operator may temporarily disable an audible alarm. User interface panel 200 may further comprise a status indicator light 230. The status indicator light may be configured such that three states are shown (1) if green, the average amplitude is between the high- and low-thresholds; (2) if red, the average amplitude is above the high-threshold; and (3) if blue, the average amplitude is below the low-threshold.

FIG. 7 shows a side panel 300 of a system according to one embodiment of the present invention. The side panel 300 comprises a power switch 310, a power jack 320 (shown connected to a power cable), and a sensor jack 330 (shown connected to a sensor cable).

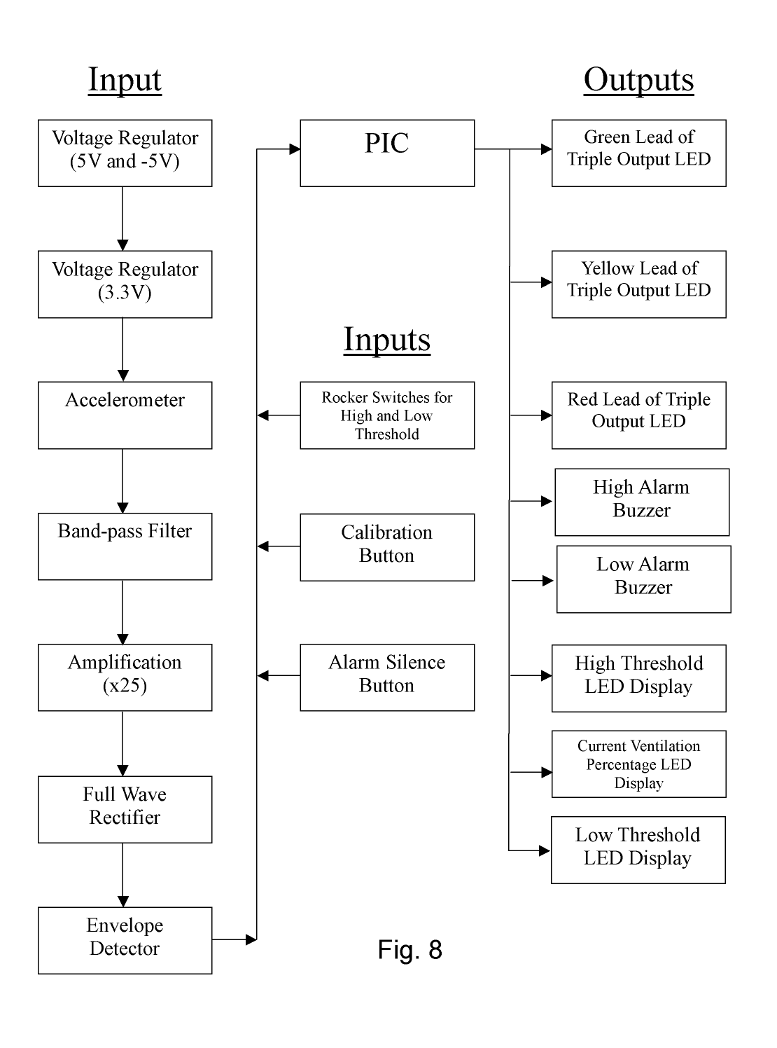

An exemplary system according to the system level diagram in FIG. 8 was built. The system was tested using a common audio speaker, driven by a sine wave, to simulate chest wall excursions. In order to reliably measure the physical displacement of the sensor, measurements were taken through the analysis of high-speed video footage: close-up videos of the vibrating sensor were shot at 120 frames per second. The maximum vibration frequency tested was 15 Hz, which falls well below the Nyquist frequency of the video's sampling rate. The path of the sensor could therefore be observed without concern for aliasing errors. However, the sampling rate was not fast enough to be sure that the peak displacements would be observable. In order to find out the peak displacement, the displacement data was run through a MATLAB program that fit a sine wave to it. The signal was fit with a sine wave because this is the known form of the vibration. The amplitude of the sine wave was then accepted as the allowing for the actual peak displacement to be determined.

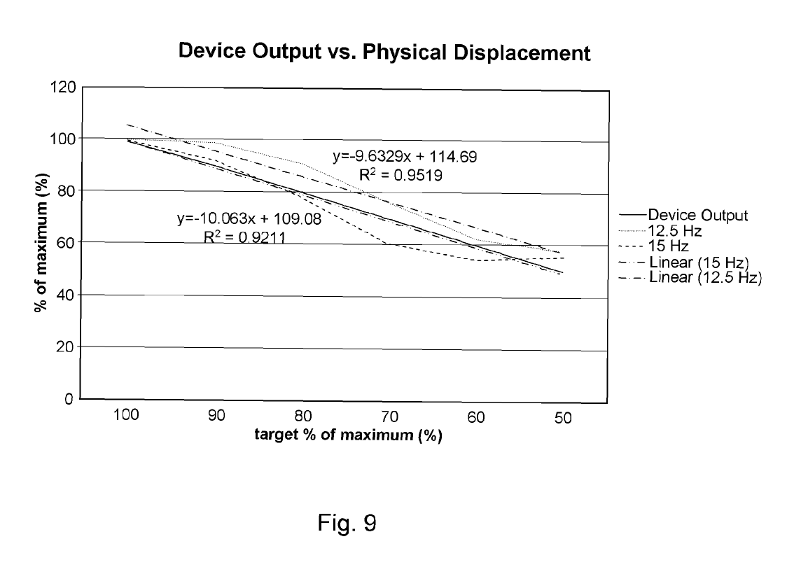

The above method was used to record the physical displacements associated with device outputs at 100%-50%, in 10% steps. The results of this comparison are illustrated in the table below and depicted graphically in FIG. 9.

| | Device output % | Displacement (mm) | Displacement % |
|---|---|---|---|
| 12.5 Hz | 100 | 0.3639 | 100 |
| | 90 | 0.3594 | 98.76339654 |
| | 80 | 0.3311 | 90.98653476 |
| | 70 | 0.2781 | 76.42209398 |
| | 60 | 0.2266 | 62.26985436 |
| | 50 | 0.2088 | 57.37840066 |
| 15 Hz | 100 | 0.4004 | 100 |
| | 90 | 0.3717 | 92.83216783 |
| | 80 | 0.3156 | 78.82117882 |
| | 70 | 0.2432 | 60.73926074 |
| | 60 | 0.2191 | 54.72027972 |
| | 50 | 0.2244 | 56.04395604 |

In FIG. 9, device output is compared with measurements obtained through high-speed video analysis, with linear best-fit lines. The ideal trend is linear, following the line y=−10x+110. The best-fit lines for the two frequencies tested were very close to the ideal line, indicating that, while there are some errors in precision, the device is functioning with a fairly high degree of accuracy and would be able to reliably detect changes in chest wall excursion.

The device was brought to the Neonatal Intensive Care Unit (“NICU”) at Strong Memorial Hospital where user tests were conducted. The NICU staff members who assisted by participating in these experiments were either registered nurses (“RN”) or respiratory therapist (“RT”). The first user test conducted assessed the visual inspection method by asking medical staff members to identify changes in the infant chest model of the test system. The user was asked to observe the test system model with the sensor in place and then look away for 20 seconds while the amplitude of the oscillating test system was modified. The amplitude was increased, decreased or remained the same. Both large changes (30% of the original) and small changes (10% of the original) were made. The following table summarizes the users' ability to identify these changes by visual inspection.

| | | Correctly Identified Change | | | | | |
|---|---|---|---|---|---|---|---|
| User | Posi- | Large | Small | Small | Large | No | |
| # | tion | Decrease | Decrease | Increase | Increase | Change | Total |
| 1 | RN | X | | | X | X | 3 |
| 2 | RN | | | X | X | | 2 |
| 3 | RN | | | X | X | | 2 |
| 4 | RN | | | X | X | | 2 |
| 5 | RT | X | | | X | | 2 |
| | | 2 | 0 | 3 | 5 | 1 | |

Of the five volunteers for the test, one correctly identified the change 3 times and the others all correctly identified the change twice. Identifying increases was easier than identifying decreases, as the only change that was correctly identified by all staff members was the large increase and the second most correctly identified change was the small increase. The small decrease was not correctly identified by any of the five users, implying that these small changes are difficult to notice by visual inspection. Only one user correctly identified when no change was made, proving that when time passes between observations, (even as small as a twenty second period) it is not easy to recall what the amplitude of oscillation looked like before.

When the staff members, who all have experience in seeing the babies on HFOV, were asked how they thought the test system model compared to a real patient on HFOV, all agreed that this was a good system both visually and functionally. It was mentioned that this system more closely correlates to a premature baby rather than a larger full term baby. Also, on a real baby, the HFOV can cause shaking in the arms and legs as well, which aids in visual inspection.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A method of monitoring high frequency oscillatory ventilation (HFOV) of an individual, comprising the steps of:
measuring oscillatory movement of the chest wall of an individual using an accelerometer;
determining an amplitude of each of a plurality of consecutive oscillations;
averaging the amplitude of the oscillations which occur during a pre-determined time span; and
comparing the average to a pre-determined baseline amplitude, wherein a variance from the pre-determined baseline causes an operator to be alerted.

2. The method of claim 1, wherein the time span is a moving time span, and the averaging and comparing steps are repeated.

3. The method of claim 1, further comprising the step of establishing a baseline amplitude when an input is received from the operator, wherein the baseline amplitude is the average amplitude at the time the input is received.

4. The method of claim 1, wherein the individual is a neonate

5. The method of claim 1, wherein the individual is an animal.

6. The method of claim 1, wherein the accelerometer is a three-axis accelerometer.

7. A system for monitoring chest wall excursion in small subjects, comprising:
an accelerometer for affixing to the chest wall of the subject;
a monitor in communication with the accelerometer, the monitor having a processing circuit configured to determine an average amplitude of an oscillatory movement of the accelerometer; and
an alarm in communication with the monitor for alerting an operator if the average amplitude varies by more than a predetermined amount.

8. The system of claim 7, wherein the accelerometer further comprises three single-axis accelerometers.

9. The system of claim 7, wherein the accelerometer is a three-axis accelerometer.

10. The system of claim 7, wherein the monitor further comprises a microcontroller.

11. The system of claim 7, wherein the monitor further comprises a digital signal processor.

12. A system for monitoring chest wall excursion in small subjects, comprising:
an accelerometer;
means for fixing the accelerometer to the body of the subject;
a monitor in communication with the accelerometer, the monitor having means for processing a signal of the accelerometer to determine an average amplitude of an oscillatory movement of the chest wall of the subject; and
means for alerting an operator if the average amplitude varies by more than a predetermined amount.
